(19) United States
(12) Patent Application Publication
(10) Pub. No.: US 2026/0027163 A1
(43) Pub. Date: Jan. 29, 2026

(54) GUT MICROBIOME BACTERIA AND ENZYMES THAT METABOLIZE DIETARY AND MEDICINAL PLANT SMALL MOLECULES TO AFFECT GUT MICROBIOME

(71) Applicant: The Children's Medical Center Corporation, Boston, MA (US)

(72) Inventors: Seth Rakoff-Nahoum, Boston, MA (US); Gavin A. Kuziel, Boston, MA (US)

(73) Assignee: The Children's Medical Center Corporation, Boston, MA (US)

(21) Appl. No.: 18/997,454

(22) PCT Filed: Jul. 21, 2023
(86) PCT No.: PCT/US2023/028354

§ 371 Date: Jan. 21, 2025

Related U.S. Application Data
(60) Provisional application No. 63/391,686, filed on Jul. 22, 2022.

(51) Int. Cl. A61K 35/74; A61K 31/7034; A61P 1/00; A61P 31/06; C12N 1/20; C12N 9/42; C12N 15/52; C12R 1/01

(52) U.S. Cl. CPC A61K 35/74; A61K 31/7034; A61P 1/00; A61P 31/06; C12N 1/20; C12N 9/2434; C12N 15/52; C12R 2001/01; C12Y 302/01

(57) ABSTRACT

The present disclosure provides pharmaceutical compositions comprising a microorganism capable of expressing one or more enzymes that convert salicin to saligenin, and salicin, or a pharmaceutically acceptable salt thereof. The present disclosure also provides methods for treating an inflammatory bowel disease (IBD) in a subject in need thereof comprising administering to the subject a therapeutically effective amount of any of the pharmaceutical compositions described herein. Methods for treating an inflammatory bowel disease (IBD) in a subject in need thereof comprising administering a therapeutically effective amount of a pharmaceutical composition comprising saligenin, or pharmaceutically acceptable salt thereof, are also provided herein. The present disclosure further provides kits, food products, nutraceuticals, and bacterial cells.

### RELATED APPLICATION

This application claims the benefit under 35 U.S.C. § 119(c) to U.S. Provisional Application No. 63/391,686, filed Jul. 22, 2022, the entire contents of which are incorporated herein by reference.

### BACKGROUND

Diet is instrumental in driving the composition and dynamics of the gut microbiome and in the development and prevention of human disease. Unlike the understanding of carbohydrate-microbe interactions, there is a dearth of knowledge as to phytochemical microbe interactions, whether these molecules are metabolized by enteric organisms, and how products of phytochemical catabolismaffect microbiome composition or host physiology. Gut symbionts may leverage distinct genetic systems to transform dietary phytochemicals into immunomodulatory metabolites, and additional study of the molecular mechanisms of dietary phytochemical metabolism by dominant gut microorganisms and the effects of this metabolism on the maintenance of intestinal homeostasis is needed to inform the development of new therapeutics.

### SUMMARY

The present disclosure describes the identification of gut bacteria and their enzymes that metabolize dietary and medicinal plant-derived chemicals into bioactive chemicals that both affect the host (e.g., attenuate autoimmune diseases, modulate immunoregulatory systems) and other gut bacteria (e.g., these metabolites kill pathogenic bacteria). As described herein, these microbes or their enzymes can be used to develop designer probiotics or personal diets for different conditions of health and disease. A number of gut bacteria species (including Bacteroides species, the dominant members in the western human gut microbiome) having previously uncharacterized proteins that metabolize chemicals that humans consume in diet and as medicine were identified. These bacteria use these enzymes to transform the chemical structures of these compounds (e.g., salicin) in a way that activates their bioactive effects. The present disclosure also describes the heterologous expression of these enzymes in other bacteria and shows that the enzymes still show activity, validating their utility for genetic engineering in other bacteria for the development of novel probiotics. The metabolites of these chemicals (e.g., saligenin) can also act on host immunoregulatory pathways (such as the Aryl Hydrocarbon Receptor, AhR). Gut or environmental bacterial enzymes that metabolize a fraction of these bioactive compounds have not been previously identified, nor have the effects of these bioactivities previously been demonstrated in vitro or in vivo. These enzymes are relatively simple and do not require other enzymes or chemical cofactors to function and can be expressed in other bacteria. The bacteria in which these enzymes are produced natively (i.e., Bacteroides species) are also the dominant bacteria in the human gut and can effectively metabolize these natural prodrugs (e.g., salicin) at the site where they are most needed (in the gut), allowing for targeted therapy of gut diseases such as inflammatory bowel disease.

Thus, in one aspect, the present disclosure provides pharmaceutical compositions comprising: (i) a microorganism capable of expressing one or more enzymes that convert salicin to saligenin, and (ii) salicin, or a pharmaceutically acceptable salt thereof.

In another aspect, the present disclosure provides methods for treating an inflammatory bowel disease (IBD) in a subject in need thereof, the method comprising administering to the subject a therapeutically effective amount of any of the pharmaceutical compositions described herein.

In another aspect, the present disclosure provides methods for treating an inflammatory bowel disease (IBD) in a subject in need thereof, the method comprising administering a therapeutically effective amount of a pharmaceutical composition comprising saligenin, or pharmaceutically acceptable salt thereof.

In another aspect, the present disclosure provides food products comprising (i) a microorganism capable of expressing one or more enzymes that convert salicin to saligenin, (ii) salicin, or a pharmaceutically acceptable salt thereof; and (iii) a food.

In another aspect, the present disclosure provides nutraceuticals comprising (i) a microorganism capable of expressing one or more enzymes that convert salicin to saligenin, (ii) salicin, or a pharmaceutically acceptable salt thereof; (iii) a food; and (iii) a dietary supplement.

In another aspect, the present disclosure provides bacterial cells comprising one or more polynucleotides encoding gshD and/or gghC from Bacteroides uniformis.

### DEFINITIONS

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “administer,” “administering,” or “administration” refers to implanting, absorbing, ingesting, injecting, inhaling, or otherwise introducing a composition described herein in or on a subject. In some embodiments, compositions described herein are administered orally (e.g., ingested).

The term “inflammatory bowel disease” (IBD) refers to a group of inflammatory conditions of the colon and small intestine. In some embodiments, IBD is Crohn's disease. In some embodiments, IBD is ulcerative colitis.

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this disclosure (e.g., salicin) include those derived from suitable inorganic and organic acids and bases. Examples of pharmaceutically acceptable, nontoxic acid addition salts are salts of an amino group formed with inorganic acids, such as hydrochloric acid, hydrobromic acid, phosphoric acid, sulfuric acid, and perchloric acid or with organic acids, such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium, and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In certain embodiments, the non-human animal is a mammal (e.g., primate (e.g., cynomolgus monkey or rhesus monkey), commercially relevant mammal (e.g., cattle, pig, horse, sheep, goat, cat, or dog), or bird (e.g., commercially relevant bird, such as chicken, duck, goose, or turkey)). In certain embodiments, the non-human animal is a fish, reptile, or amphibian. The non-human animal may be a male or female at any stage of development. The non-human animal may be a transgenic animal or genetically engineered animal. The term “patient” refers to a human subject in need of treatment of a disease. In some embodiments, a subject has an inflammatory bowel disease or is suspected of having an inflammatory bowel disease (e.g., Crohn's disease or ulcerative colitis).

A “therapeutically effective amount” of a composition described herein is an amount sufficient to provide a therapeutic benefit in the treatment of a condition (e.g., an inflammatory bowel disease) or to delay or minimize one or more symptoms associated with the condition. A therapeutically effective amount of a composition means an amount of therapeutic agent, alone or in combination with other therapies, which provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent. In certain embodiments, a therapeutically effective amount is an amount sufficient for activating aryl hydrocarbon receptor (AhR). In certain embodiments, a therapeutically effective amount is an amount sufficient for treating an inflammatory bowel disease (e.g., ulcerative colitis or Crohn's disease).

The terms “treatment,” “treat,” and “treating” refer to reversing, alleviating, delaying the onset of, or inhibiting the progress of a disease described herein, such as an inflammatory bowel disease. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms and/or in light of exposure to a pathogen). Treatment may also be continued after symptoms have resolved, for example, to delay or prevent recurrence.

### BRIEF DESCRIPTION OF THE DRAWINGS

FIGS. 1A-1B show that saligenin inhibited production of anti-inflammatory cytokines. Saligenin pre-treatment of macrophages stimulated with pro-inflammatory LP5 attenuated inflammatory cytokine production in vitro.

FIG. 2 shows a scheme demonstrating the biomechanism of converting salicin to saligenin by B. uniformis.

FIG. 3 shows the chemical structures of salicin metabolites saligenin, salicylic acid, and acetylsalicylic acid.

FIG. 4 shows that saligenin activated the pro-homeostatic xenobiotic sensor AhR. Saligenin antagonized AhR, a xenobiotic immune sensor that drives pro-homeostatic processes in gut epithelia and immune cells.

FIG. 5 shows schemes demonstrating the therapeutic effects of salicin and its metabolites.

FIGS. 6A-6B show that saligenin-induced protection from DSS correlated with a decrease in Th1-effector cells (T-cell subset most associated with Crohn's disease in humans).

FIGS. 7A-7B show that bioactivation of salicin to saligenin by Bacteroides uniformis protected against DSS-induced colitis.

FIGS. 8A-8B show that the salicin derivative saligenin protected against DSS-induced intestinal inflammation as pure compound delivered orally daily for 7 days (in mice).

FIG. 9 shows that saligenin activation of AhR in vivo can be reversed with a competitive AhR antagonist, confirming saligenin-mediated protection from colitis acted through a novel mechanism-of-action of AhR activation.

FIG. 10 shows modes of bacteria/salicin or saligenin delivery.

FIGS. 11A-11B show that saligenin-induced protection from DSS correlated with a decrease in colonic IFN-gamma (an inflammatory factor associated with IBD).

FIG. 12 shows the mechanism of action of saligenin-induced protection from colitis. Saligenin may be acting on AhR, a next-generation immune receptor that recognizes small molecules in the gut and modulates downstream immunomodulatory pathway. Agonism of AhR may inhibit pro-inflammatory cytokine production. In certain embodiments, saligenin may be developed as an orally available small molecule therapeutic for mild-to-moderate IBD as an alternative to (1) more burdensome/expensive biologics treatments (e.g., anti-TNF) to which not all patients respond or (2) last-generation anti-inflammatory molecules like 5-ASA or corticosteroids that are not pan-effective in maintaining discase remission.

FIGS. 13A-13B show schematics of methods used to determine the scope of phytochemical metabolism across prominent human gut bacteria utilizing techniques within microbiology and culturomics; identify and characterize the genetic and enzymatic basis for phytochemical metabolism, leveraging techniques within microbial genetics and molecular biochemistry; and assess the pro-or anti-homeostatic effects of phytochemical metabolism on host physiology using coupled in vitro and in vivo models of intestinal disease such as colitis, pathogen infection, or colorectal carcinogenesis.

FIG. 14 shows that taxonomically broad gut bacteria metabolized diverse phytochemicals.

FIG. 15 shows that species-and chemical-level variation dominated Bacteroides phytochemical metabolism.

FIGS. 16A-16D show that two prominent gut Bacteroides species leveraged distinct genetic systems for phytochemical metabolism.

FIGS. 17A-17B show that Bacteroides phytochemical metabolism drove intestinal homeostasis in experimental colitis.

### DETAILED DESCRIPTION

Delivery of salicin plus Bacteroides as a synbiotic therapeutic is a novel therapeutic modality for targeted activation of a medicinal small molecule (salicin) in the colon where the Bacteroides are predominant, (1) lessening the need to dose at high levels to saturate system and (2) potentially preventing off-target effects.

Engineering of any Bacteroides with enzymes specific to novel salicin derivatives would enable targeted metabolism of distinct substrates in the lower gut depending on probiotic consumed, allowing for combinatorial activation of anti-IBD small molecules with potentially different mechanisms of action/effects on different immune cell subsets.

Bacteroides as probiotic scaffolds are appealing as they are (1) oxygen-resistant and genetically tractable organisms, they (2) grow on diverse carbohydrates (enabling potential engraftment on specific diets) and they are (3) dominant members of the natural gut microbiome (unlike E. coli, for example), suggesting they might colonize the gut more robustly/resiliently over time, requiring less regular dosing over time.

Thus, the present disclosure provides a novel suite of bacterial and enzymatic deliverables with direct translatability as synbiotic therapeutics to treat a range of intestinal disorders (colitis, infection, etc.) relevant to warfighters operating in endemic regions of the world.

### Pharmaceutical Compositions and Kits

In one aspect, the present disclosure provides pharmaceutical compositions comprising (i) a microorganism capable of expressing one or more enzymes that convert salicin to saligenin (e.g., gshD and/or gghC from Bacteroides uniformis), and (ii) salicin, or a pharmaceutically acceptable salt thereof. In some embodiments, a pharmaceutical composition further comprises a pharmaceutically acceptable carrier or buffer (e.g., a pharmaceutically acceptable excipient). In some embodiments, a pharmaceutical composition is formulated to dissolve in the digestive or GI tract of a subject. In some embodiments, a pharmaceutical composition is formulated to dissolve at a pH of about 6.8.

Various microorganisms may be included in the pharmaceutical compositions described herein. In some embodiments, the microorganism is bacteria. In certain embodiments, the microorganism is not genetically engineered to express the one or more enzymes that convert salicin to saligenin. In some embodiments, the microorganism is a species of Bacteroides (e.g., Bacteroides uniformis). In certain embodiments, the microorganism is genetically engineered to heterologously express the one or more enzymes that convert salicin to saligenin. In some embodiments, the microorganism is lyophilized.

In some embodiments, a pharmaceutical composition is a pill, tablet, syrup, or solution. In some embodiments, a pharmaceutical composition is a pill or tablet. In some embodiments, a pharmaceutical composition is a pill. In some embodiments, a pharmaceutical composition is a tablet. In some embodiments, a pharmaceutical composition is capable of local delivery to the digestive or GI tract of a subject. In some embodiments, a pharmaceutical composition comprises one or more pharmaceutically acceptable excipients that chemically and/or structurally predispose the pharmaceutical composition for delivery to the digestive or GI tract of a subject.

In some embodiments, the microorganism converts the salicin to saligenin. In certain embodiments, the microorganism does not convert the salicin to saligenin until the pharmaceutical composition has been delivered to the digestive or GI tract of a subject.

In certain embodiments, a pharmaceutical composition described herein is provided in an effective amount to a subject. In certain embodiments, the effective amount is a therapeutically effective amount. In certain embodiments, the effective amount is a prophylactically effective amount. In certain embodiments, the effective amount is an amount effective for treating an inflammatory bowel disease in a subject in need thereof. In certain embodiments, the effective amount is an amount effective for preventing the development of an inflammatory bowel disease in a subject in need thereof. In certain embodiments, the effective amount is an amount effective for reducing the risk of developing an inflammatory bowel disease in a subject in need thereof.

In certain embodiments, the subject is an animal. The animal may be of either sex and may be at any stage of development. In certain embodiments, the subject described herein is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

Pharmaceutical compositions described herein can be prepared by any method known in the art of pharmaceutics. In general, such preparatory methods include bringing the components of the composition described herein (i.e., the “active ingredients”) into association with a carrier or excipient, and/or one or more other accessory ingredients, and then, if necessary and/or desirable, shaping, and/or packaging the product into a desired single-or multi-dose unit.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly (vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, ascorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip®, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredients can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Pharmaceutical compositions provided herein are typically formulated in dosage unit form for case of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a pharmaceutical composition required to achieve an effective amount will vary from subject to subject, depending, for example, on species, age, and general condition of a subject, severity of the side effects or disorder, identity of the particular composition, mode of administration, and the like. An effective amount may be included in a single dose (e.g., single oral dose) or multiple doses (e.g., multiple oral doses). In certain embodiments, when multiple doses are administered to a subject or applied to a tissue or cell, any two doses of the multiple doses include different or substantially the same amounts of a composition described herein. In certain embodiments, when multiple doses are administered to a subject or applied to a tissue or cell, the frequency of administering the multiple doses to the subject or applying the multiple doses to the tissue or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the tissue or cell is one dose per day. In certain embodiments, the frequency of administering the multiple doses to the subject or applying the multiple doses to the tissue or cell is two doses per day. In certain embodiments, the frequency of administering the multiple doses to the subject or applying the multiple doses to the tissue or cell is three doses per day. In certain embodiments, when multiple doses are administered to a subject or applied to a tissue or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, tissue, or cell. In certain embodiments, the duration between the first dose and last dose of the multiple doses is three months, six months, or one year. In certain embodiments, the duration between the first dose and last dose of the multiple doses is the lifetime of the subject, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a composition described herein. In certain embodiments, a dose described herein includes independently between 1 mg and 3 mg, inclusive, of a composition described herein. In certain embodiments, a dose described herein includes independently between 3 mg and 10 mg, inclusive, of a composition described herein. In certain embodiments, a dose described herein includes independently between 10 mg and 30 mg, inclusive, of a composition described herein. In certain embodiments, a dose described herein includes independently between 30 mg and 100 mg, inclusive, of a composition described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A composition, as described herein, can be administered in combination with one or more additional pharmaceutical agents (e.g., therapeutically and/or prophylactically active agents). The compositions can be administered in combination with additional pharmaceutical agents that improve their activity (e.g., activity (e.g., potency and/or efficacy) in treating a disease in a subject in need thereof, in preventing a disease in a subject in need thereof, in reducing the risk to develop a disease in a subject in need thereof, and/or in inhibiting the activity of a protein kinase in a subject or cell), improve bioavailability, improve safety, reduce drug resistance, reduce and/or modify metabolism, inhibit excretion, and/or modify distribution in a subject or cell. It will also be appreciated that the therapy employed may achieve a desired effect for the same disorder, and/or it may achieve different effects. In certain embodiments, a pharmaceutical composition described herein including a compound described herein and an additional pharmaceutical agent shows a synergistic effect that is absent in a pharmaceutical composition including one of the compound and the additional pharmaceutical agent, but not both. In some embodiments, the additional pharmaceutical agent achieves a desired effect for the same disorder. In some embodiments, the additional pharmaceutical agent achieves different effects.

The composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease, hematological disease, neurological disease, painful condition, psychiatric disorder, or metabolic disorder). Each additional pharmaceutical agent may be administered at a dose and/or on a time schedule determined for that pharmaceutical agent. The additional pharmaceutical agents may also be administered together with each other and/or with the composition described herein in a single dose or composition or administered separately in different doses or compositions. The particular combination to employ in a regimen will take into account compatibility of the composition described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

Also encompassed by the disclosure are kits (e.g., pharmaceutical packs). The kits provided may comprise a pharmaceutical composition described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition described herein. In some embodiments, the pharmaceutical composition described herein provided in the first container and the second container are combined to form one unit dosage form.

Thus, in one aspect, provided are kits including a first container comprising a pharmaceutical composition described herein. In certain embodiments, the kits are useful for treating a disease (e.g., an inflammatory bowel disease) in a subject in need thereof. In certain embodiments, the kits are useful for preventing a disease (e.g., an inflammatory bowel disease) in a subject in need thereof. In certain embodiments, the kits are useful for reducing the risk of developing a disease (e.g., an inflammatory bowel disease) in a subject in need thereof.

In certain embodiments, a kit described herein further includes instructions for using the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., an inflammatory bowel disease) in a subject in need thereof. In certain embodiments, the kits and instructions provide for preventing a disease (e.g., an inflammatory bowel diseae) in a subject in need thereof. In certain embodiments, the kits and instructions provide for reducing the risk of developing a disease (e.g., an inflammatory bowel disease) in a subject in need thereof. A kit described herein may include one or more additional pharmaceutical agents described herein as a separate composition.

### Methods for Treating an Inflammatory Bowel Disease

In another aspect, the present disclosure provides methods for treating an inflammatory bowel disease (IBD) in a subject in need thereof, the method comprising administering to the subject a therapeutically effective amount of any of the pharmaceutical compositions provided herein. In some embodiments, the IBD is Crohn's disease. In some embodiments, the IBD is ulcerative colitis. In some embodiments, the subject is a human. In some embodiments, the pharmaceutical composition is administered to the subject orally. In some embodiments, the treatment of IBD is effected through aryl hydrocarbon receptor (AhR) agonism.

In another aspect, the present disclosure provides methods for treating an inflammatory bowel disease (IBD) in a subject in need thereof, the method comprising administering a therapeutically effective amount of a pharmaceutical composition comprising saligenin, or pharmaceutically acceptable salt thereof. In some embodiments, the pharmaceutical composition further comprises a pharmaceutically acceptable carrier or buffer. In certain embodiments, the IBD is Crohn's disease. In certain embodiments, the IBD is ulcerative colitis. In some embodiments, the pharmaceutical composition is administered as a pill, tablet, syrup, or solution. In some embodiments, the subject is a human. In some embodiments, the pharmaceutical composition is administered to the subject orally. In certain embodiments, the pharmaceutical composition is capable of local delivery to the digestive or GI tract. In certain embodiments, the pharmaceutical composition is formulated to dissolve in the digestive or GI tract. In certain embodiments, the pharmaceutical composition is formulated to dissolve at a pH of about 6.8. In some embodiments, the treatment of IBD is effected through aryl hydrocarbon receptor (AhR) agonism.

### Food Products, Nutraceuticals, and Bacterial Cells

In other aspects, the present disclosure provides food products, nutraceuticals, and bacterial cells.

In one aspect, the present disclosure provides food products comprising (i) a microorganism capable of expressing one or more enzymes that convert salicin to saligenin, (ii) salicin, or a pharmaceutically acceptable salt thereof; and (iii) a food.

In another aspect, the present disclosure provides nutraceuticals comprising (i) a microorganism capable of expressing one or more enzymes that convert salicin to saligenin, (ii) salicin, or a pharmaceutically acceptable salt thereof; (iii) a food; and (iii) a dietary supplement.

In another aspect, the present disclosure provides bacterial cells comprising one or more polynucleotides encoding gshD and/or gghC from Bacteroides uniformis.

### EXAMPLES

In order that the present disclosure may be more fully understood, examples are set forth. The examples described in this application are offered to illustrate the methods, products, and compositions provided herein and are not to be construed in any way as limiting their scope.

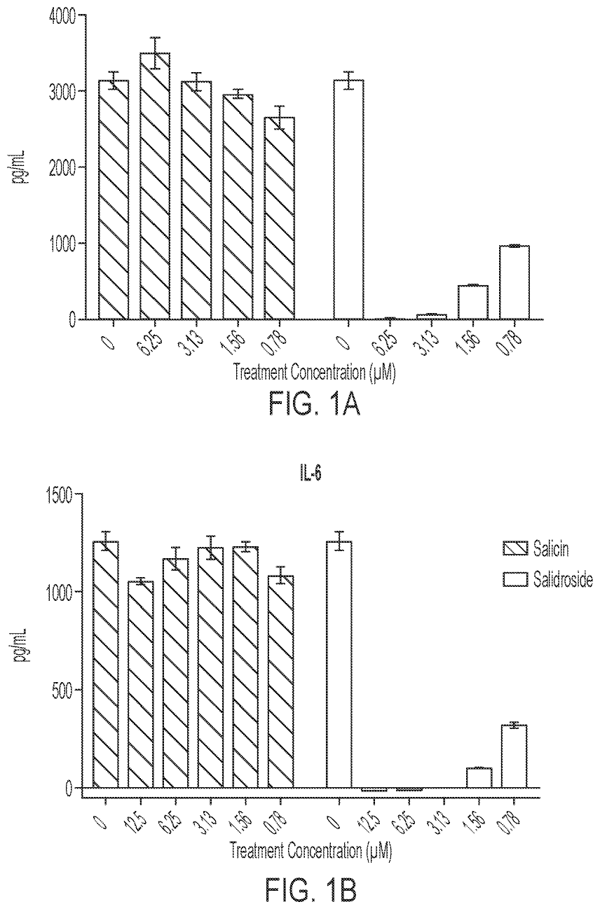

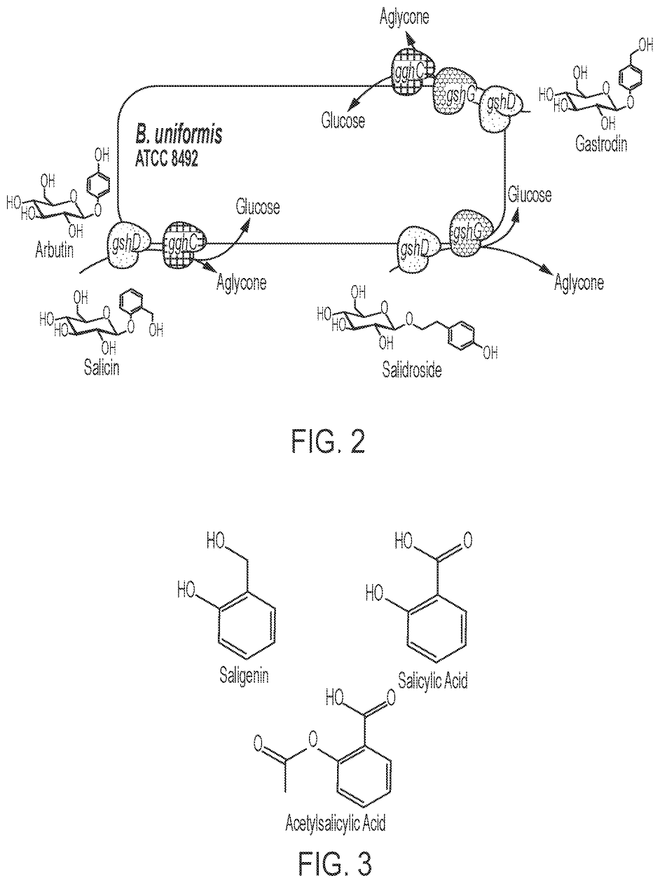

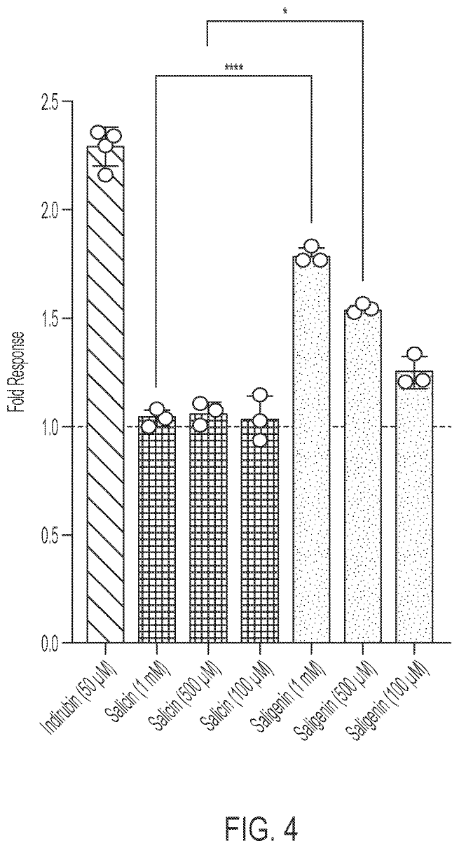

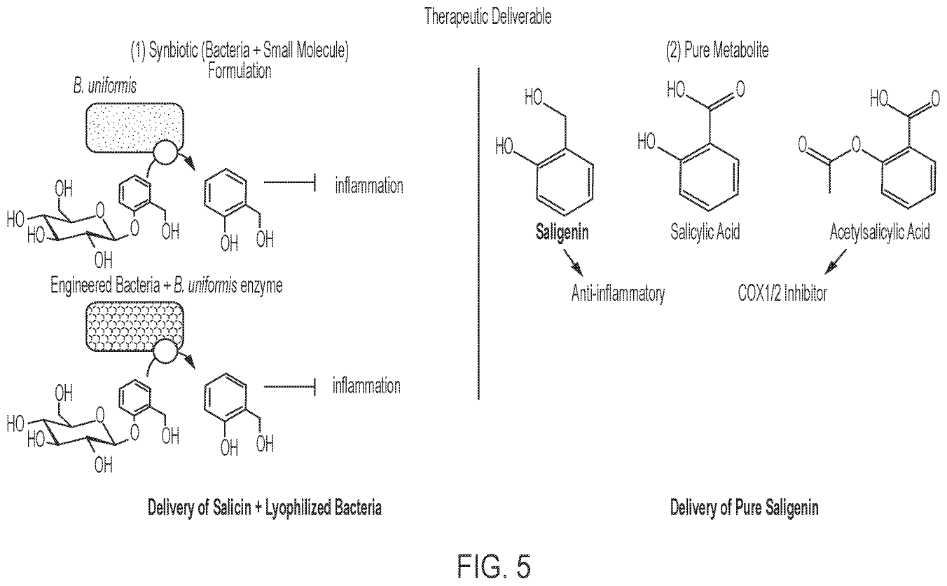

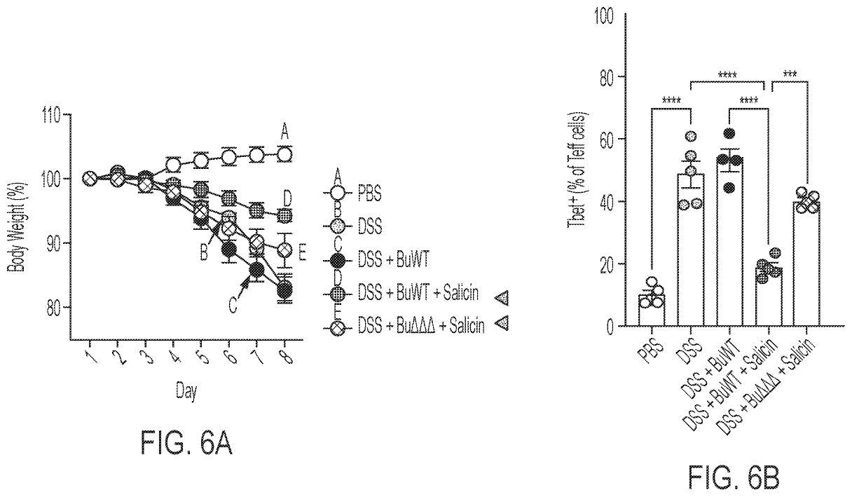

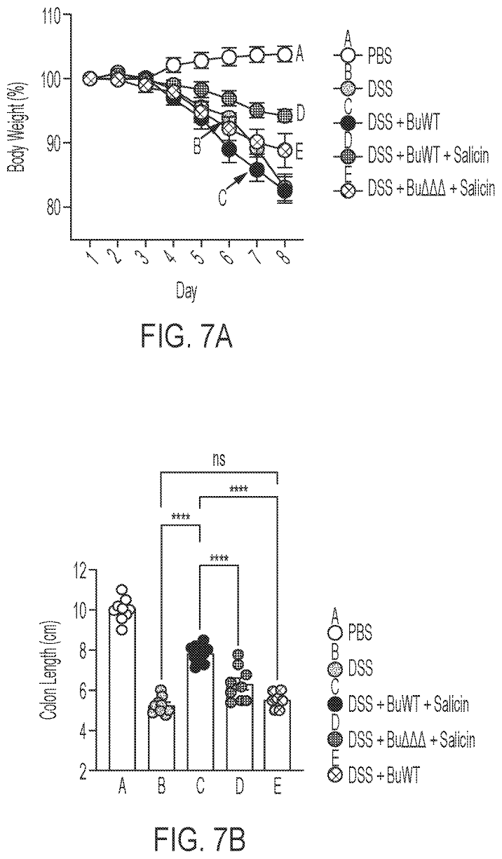

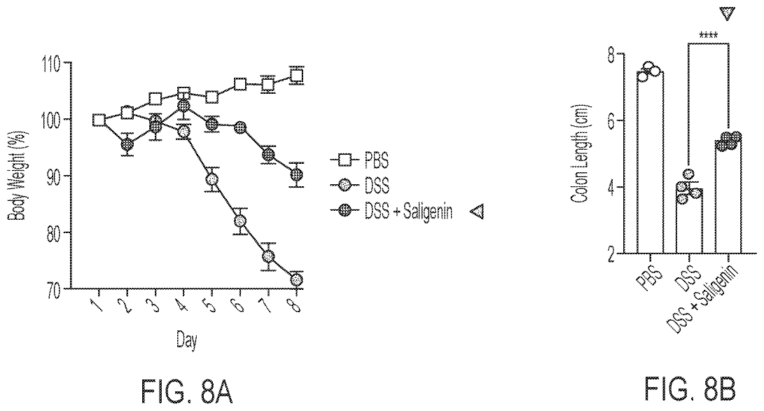

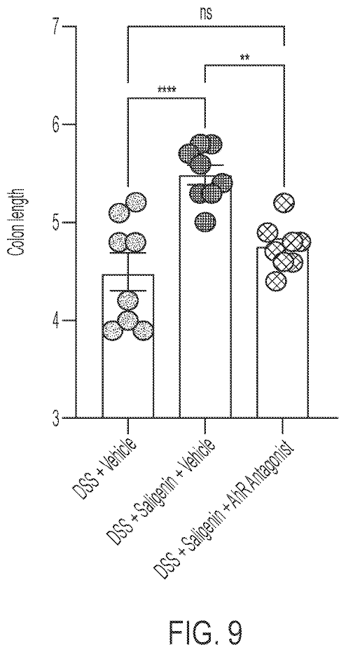

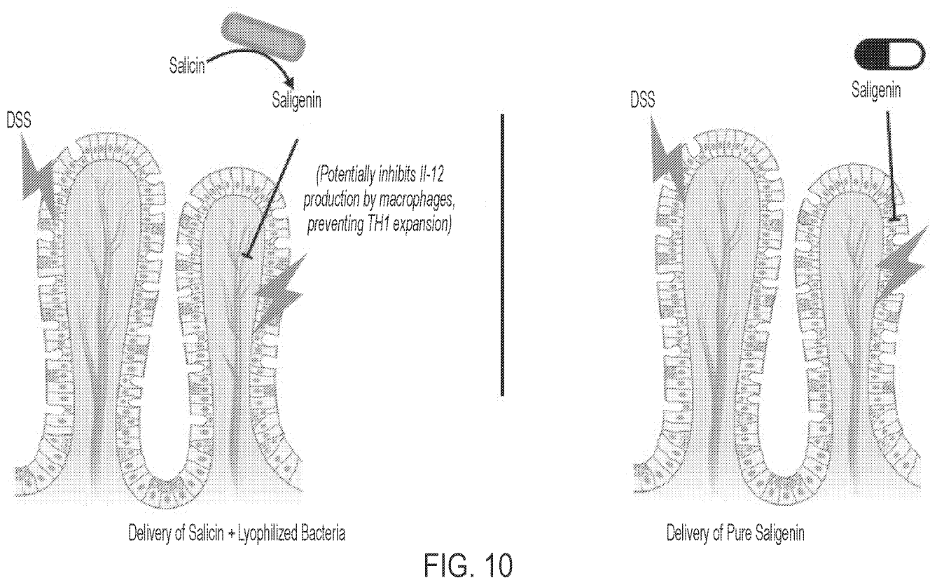

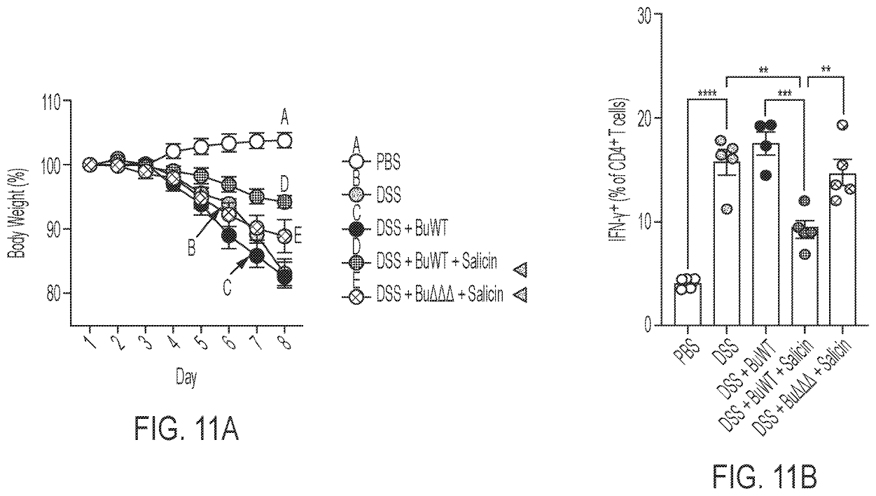

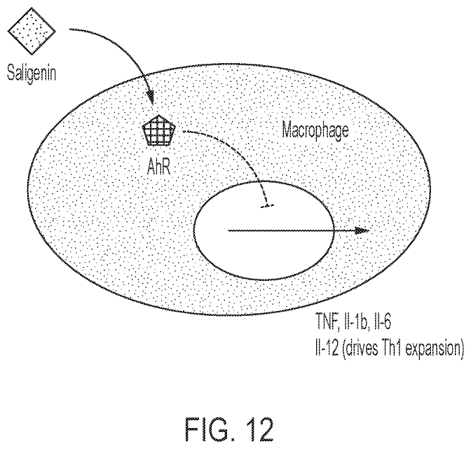

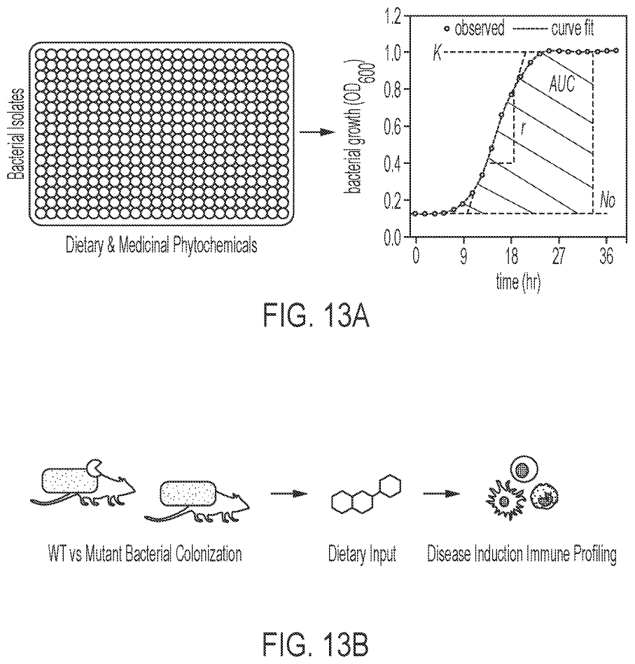

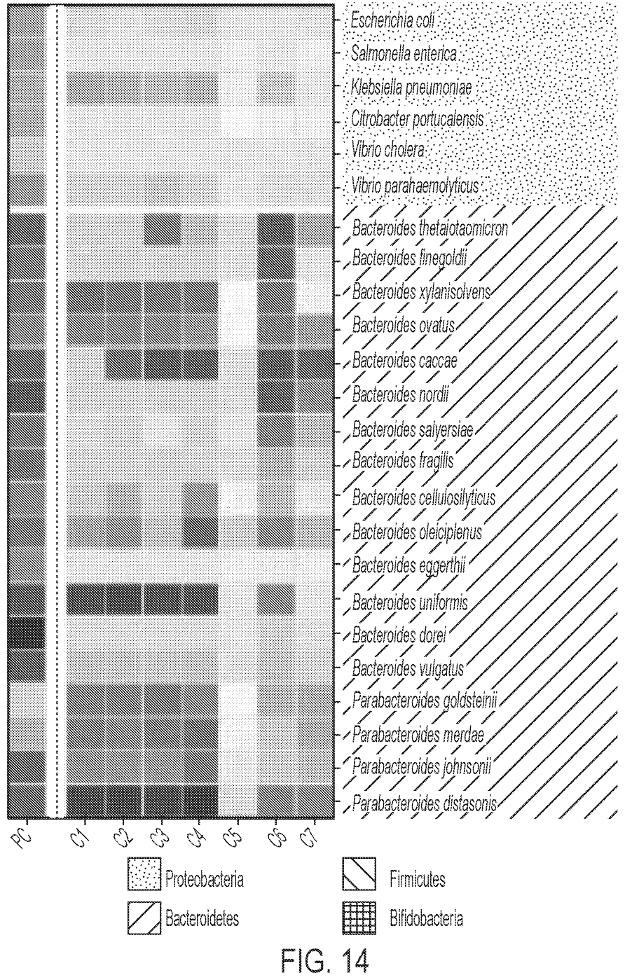

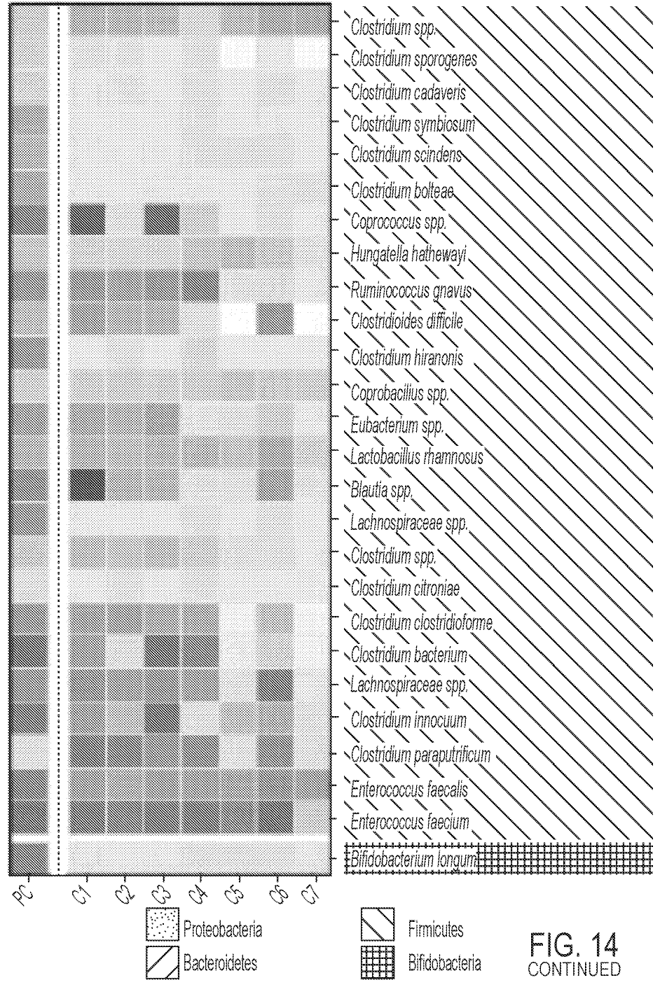

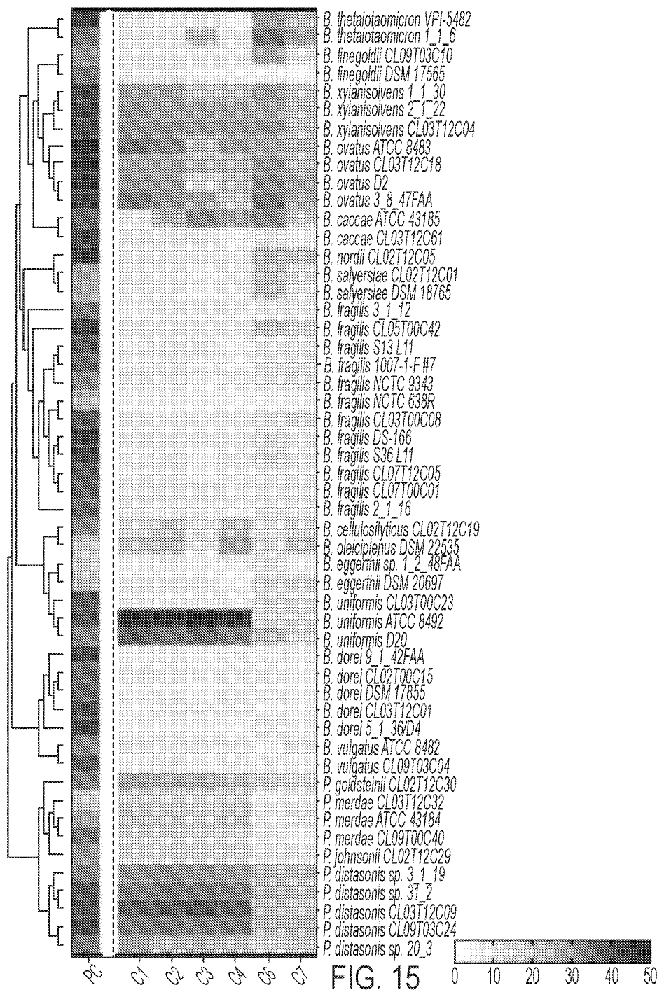

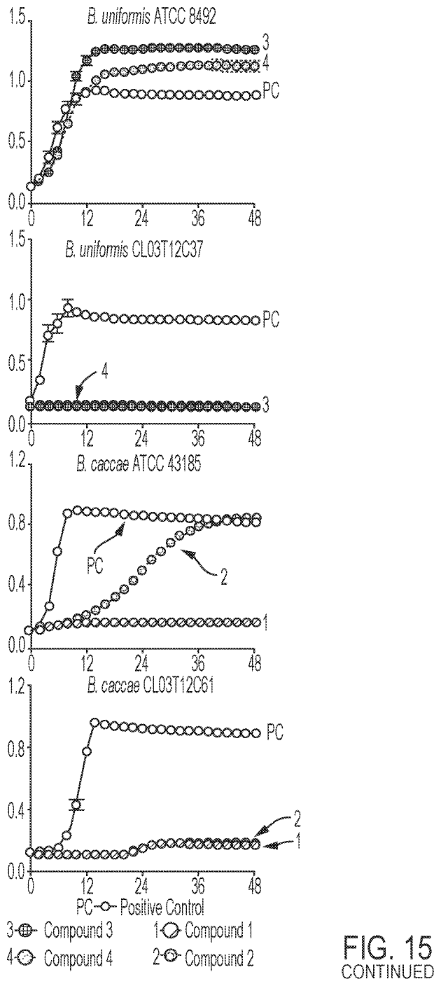

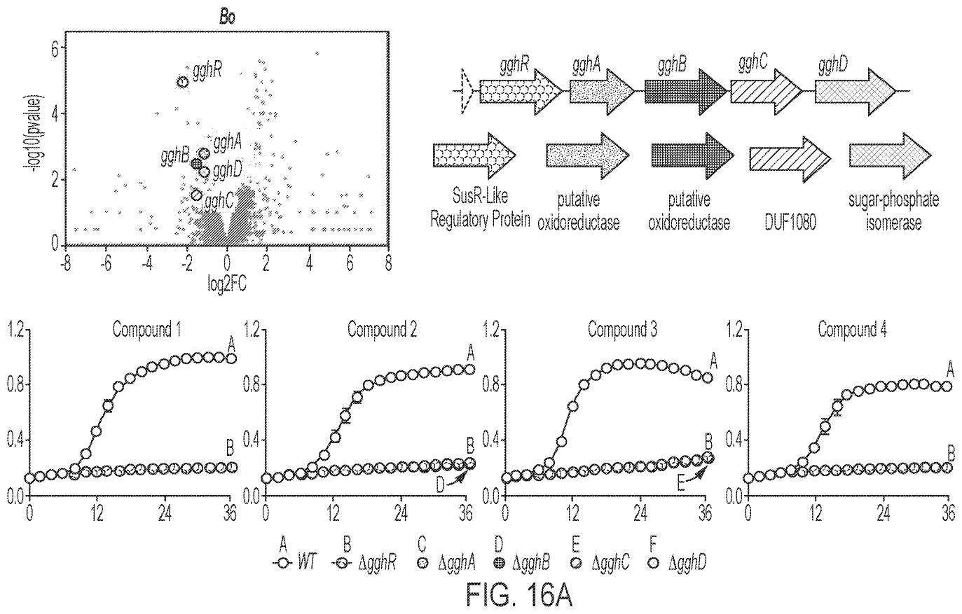

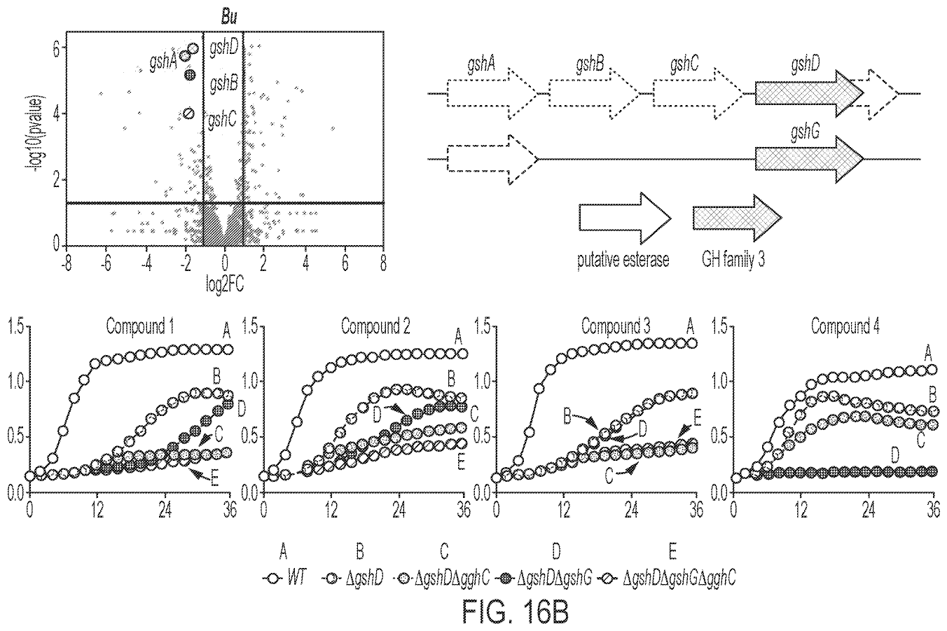

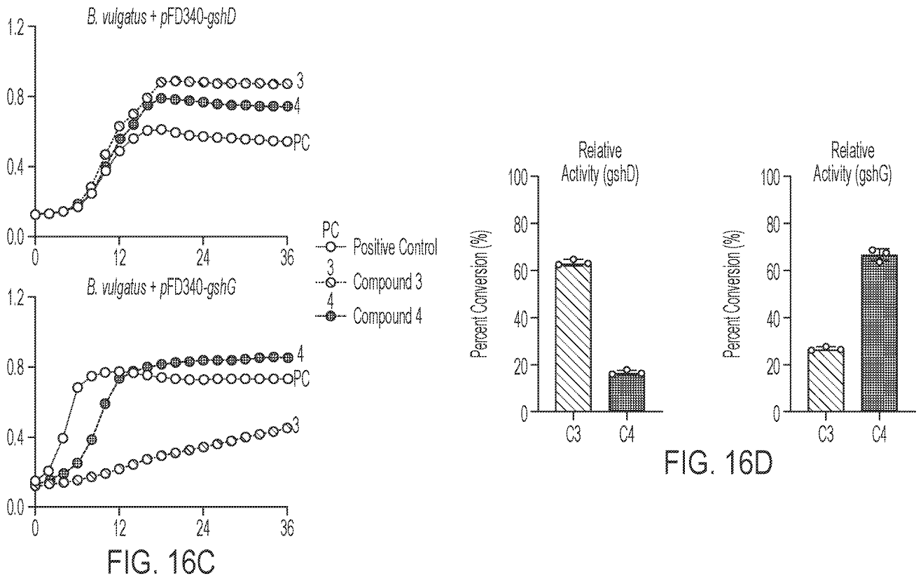

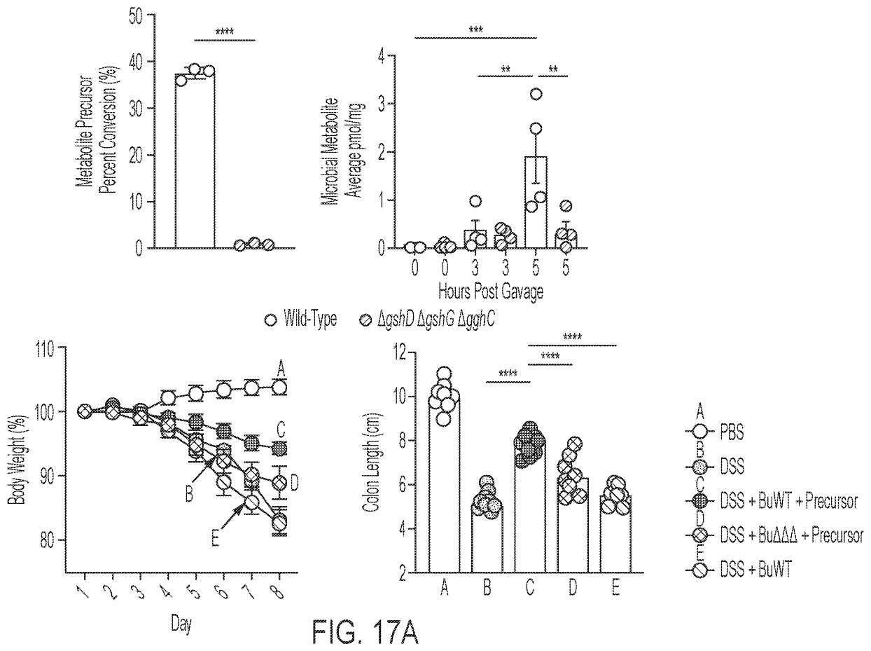

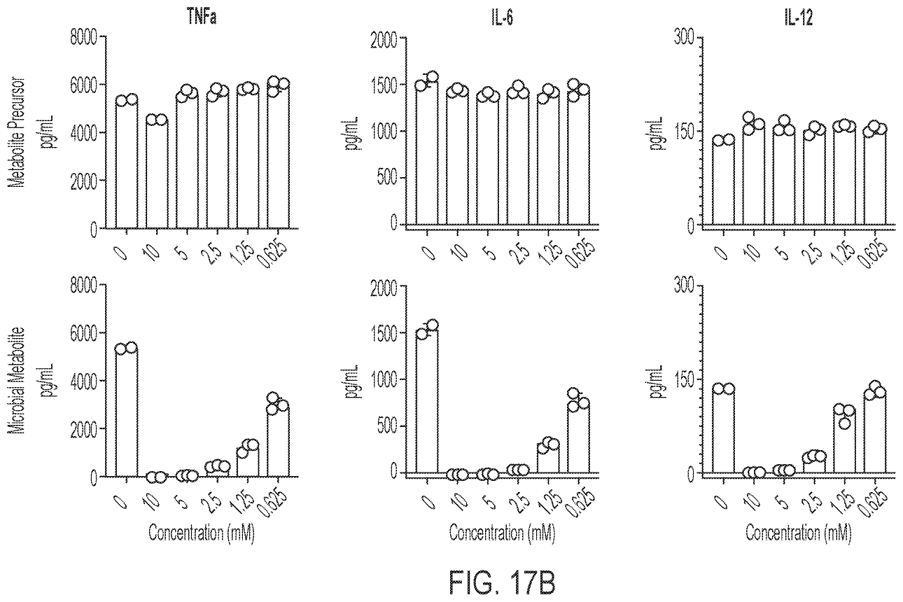

The present disclosure identifies and characterizes a novel mechanism by which the gut microbiome transforms abundant and chemically diverse small molecule components of human diet to drive pro-homeostatic programs in the intestine. Experiments demonstrating and enabling the practice of the subject matter of the present disclosure are described in FIGS. 1-19. For experiments based on dextran sulfate sodium (DSS) induced colitis (e.g., FIGS. 6-11), less weight loss during DSS exposure suggests protection, longer colon length post DSS exposure suggests protection, and fewer Th1/Th17 T-cells suggests protection.

### Equivalents and Scope

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

What is claimed is:

1. A pharmaceutical composition comprising:
(i) a microorganism capable of expressing one or more enzymes that convert salicin to saligenin, and
(ii) salicin, or a pharmaceutically acceptable salt thereof.

2. The pharmaceutical composition of claim 1 further comprising a pharmaceutically acceptable carrier or buffer.

3. The pharmaceutical composition of claim 1 or 2, wherein the pharmaceutical composition is formulated to dissolve in the digestive or GI tract of a subject.

4. The pharmaceutical composition of any one of claims 1-3, wherein the pharmaceutical composition is formulated to dissolve at a pH of about 6.8.

5. The pharmaceutical composition of any one of claims 1-4, wherein the microorganism is bacteria.

6. The pharmaceutical composition of any one of claims 1-5, wherein the microorganism is not genetically engineered to express the one or more enzymes that convert salicin to saligenin.

7. The pharmaceutical composition of any one of claims 1-6, wherein the microorganism is a species of Bacteroides.

8. The pharmaceutical composition of any one of claims 1-7, wherein the microorganism is Bacteroides uniformis.

9. The pharmaceutical composition of any one of claims 1-8, wherein the microorganism is genetically engineered to heterologously express the one or more enzymes that convert salicin to saligenin.

10. The pharmaceutical composition of any one of claims 1-9, wherein the microorganism is lyophilized.

11. The pharmaceutical composition of any one of claims 1-10, wherein the one or more enzymes that convert salicin to saligenin comprise gshD and/or gghC from Bacteroides uniformis.

12. The pharmaceutical composition of any one of claims 1-11, wherein the pharmaceutical composition is a pill, tablet, syrup, or solution.

13. The pharmaceutical composition of any one of claims 1-12, wherein the pharmaceutical composition is capable of local delivery to the digestive or GI tract of a subject.

14. The pharmaceutical composition of any one of claims 1-13, wherein the pharmaceutical composition comprises one or more pharmaceutically acceptable excipients that chemically and/or structurally predispose the pharmaceutical composition for delivery to the digestive or GI tract of a subject.

15. The pharmaceutical composition of any one of claims 1-14, wherein the microorganism converts the salicin to saligenin.

16. The pharmaceutical composition of claim 15, wherein the microorganism does not convert the salicin to saligenin until the pharmaceutical composition has been delivered to the digestive or GI tract of a subject.

17. A method for treating an inflammatory bowel disease (IBD) in a subject in need thereof, the method comprising administering to the subject a therapeutically effective amount of the pharmaceutical composition of claim 1.

18. The method of claim 17, wherein the IBD is Crohn's disease.

19. The method of claim 17, wherein the IBD is ulcerative colitis.

20. The method of any one of claims 17-19, wherein the subject is a human.

21. The method of any one of claims 17-20, wherein the pharmaceutical composition is administered to the subject orally.

22. The method of any one of claims 17-21, wherein the treatment of IBD is effected through aryl hydrocarbon receptor (AhR) agonism.

23. The pharmaceutical composition of any one of claims 1-16 for use in treating an inflammatory bowel disease (IBD).

24. The pharmaceutical composition of claim 23, wherein the IBD is Crohn's disease.

25. The pharmaceutical composition of claim 23, wherein the IBD is ulcerative colitis.

26. The pharmaceutical composition of any one of claims 23-25, wherein the treatment of IBD is effected through aryl hydrocarbon receptor (AhR) agonism.

27. A kit comprising the pharmaceutical composition of any one of claims 1-16.

28. A food product comprising:
(i) a microorganism capable of expressing one or more enzymes that convert salicin to saligenin,
(ii) salicin, or a pharmaceutically acceptable salt thereof; and
(iii) a food.

29. A nutraceutical comprising:
(i) a microorganism capable of expressing one or more enzymes that convert salicin to saligenin,
(ii) salicin, or a pharmaceutically acceptable salt thereof;
(iii) a food; and
(iii) a dietary supplement.

30. A bacterial cell comprising one or more polynucleotides encoding gshD and/or gghC from Bacteroides uniformis.

31. A method for treating an inflammatory bowel disease (IBD) in a subject in need thereof, the method comprising administering a therapeutically effective amount of a pharmaceutical composition comprising saligenin, or pharmaceutically acceptable salt thereof.

32. The method of claim 31, wherein the pharmaceutical composition further comprises a pharmaceutically acceptable carrier or buffer.

33. The method of claim 31 or 32, wherein the IBD is Crohn's disease.

34. The method of claim 31 or 32, wherein the IBD is ulcerative colitis.

35. The method of any one of claims 31-34, wherein the pharmaceutical composition is administered as a pill, tablet, syrup, or solution.

36. The method of any one of claims 31-35, wherein the subject is a human.

37. The method of any one of claims 31-36, wherein the pharmaceutical composition is administered to the subject orally.

38. The method of any one of claims 31-37, wherein the pharmaceutical composition is capable of local delivery to the digestive or GI tract.

39. The method of any one of claims 31-38, wherein the pharmaceutical composition is formulated to dissolve in the digestive or GI tract.

40. The method of any one of claims 31-39, wherein the pharmaceutical composition is formulated to dissolve at a pH of about 6.8.

41. The method of any one of claims 31-40, wherein the treatment of IBD is effected through aryl hydrocarbon receptor (AhR) agonism.
